SEQUENCE LISTING (18 sequences)

SEQ ID NO: 1 - DNA, 19 bp, Homo sapiens
gctggtcatcctcatcctg

SEQ ID NO: 2 - DNA, 19 bp, Homo sapiens
agatgtcagtcatgctctt

SEQ ID NO: 3 - protein, 1071 aa, Artificial Sequence
  CCR5-Uco-L
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNHGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNHGGKQALETVQRLLPVLCQAHGLTPEQVVAIASNHGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIVSNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 4 - protein, 1071 aa, Artificial Sequence
  CCR5-Uco-R
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNHGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNHGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 5 - protein, 34 aa, Artificial Sequence
  TALEN repeat
LTPXQVVAIXSXXGGKQALETVQRLLPVLCQXHG

SEQ ID NO: 6 - protein, 34 aa, Artificial Sequence
  TALEN Repeat
LTPEQVVAIASXXGGKQALETVQRLLPVLCQAHG

SEQ ID NO: 7 - protein, 34 aa, Artificial Sequence
  TALEN Repeat
LTPQQVVAIASXXGGKQALETVQRLLPVLCQAHG

SEQ ID NO: 8 - protein, 34 aa, Artificial Sequence
  TALEN Repeat
LTPDQVVAIASXXGGKQALETVQRLLPVLCQDHG

SEQ ID NO: 9 - protein, 34 aa, Artificial Sequence
  TALEN Repeat
LTPAQVVAIASXXGGKQALETVQRLLPVLCQDHG

SEQ ID NO: 10 - protein, 34 aa, Artificial Sequence
  TALEN Repeat
LTPEQVVAIVSXXGGKQALETVQRLLPVLCQAHG

SEQ ID NO: 11 - DNA, 1059 bp, Homo sapiens
  hCCR5 DNA
atggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtga

SEQ ID NO: 12 - protein, 198 aa, Planomicrobium okeanokoites
  FokI cleavage domain
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 13 - protein, 1037 aa, Artificial Sequence
  CCR5-Mco-L
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 14 - protein, 1037 aa, Artificial Sequence
  CCR5-Mco-R
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 15 - DNA, 18 bp, Homo sapiens
gtgggcaacatgctggtc

SEQ ID NO: 16 - protein, 18 aa, Homo sapiens
CTTCAGCCTTTTGCAGTT

SEQ ID NO: 17 - RNA, 3788 nt, Artificial Sequence
  CCR5-Uco-L mRNA
ggggccaccauggacuauaaggaccacgacggagacuacaaggaucaugauauugauuacaaagacgaugacgauaagauggccccaaagaagaagcggaaggucgguauccacggagucccagcagccguagauuugagaacuuugggauauucacagcagcagcaggaaaagaucaagcccaaagugaggucgacagucgcgcagcaucacgaagcgcuggugggucauggguuuacacaugcccacaucguagccuugucgcagcacccugcagcccuuggcacggucgccgucaaguaccaggacaugauugcggcguugccggaagccacacaugaggcgaucgucgguguggggaaacaguggagcggagcccgagcgcuugaggcccuguugacggucgcgggagagcugagagggccuccccuucagcuggacacgggccaguugcugaagaucgcgaagcggggaggagucacggcggucgaggcggugcacgcguggcgcaaugcgcucacgggagcaccccucaaccugaccccagagcaggucguggcaauugcgagcaaccacgggggaaagcaggcacucgaaaccguccagagguugcugccugugcugugccaagcgcacggacuuacgccagagcaggucguggcaauugcgagccaugacgggggaaagcaggcacucgaaaccguccagagguugcugccugugcugugccaagcgcacggacuaaccccagagcaggucguggcaauugcgagcaacggagggggaaagcaggcacucgaaaccguccagagguugcugccugugcugugccaagcgcacggguugaccccagagcaggucguggcaauugcgagcaaccacgggggaaagcaggcacucgaaaccguccagagguugcugccugugcugugccaagcgcacggccugaccccagagcaggucguggcaauugcgagcaaccacgggggaaagcaggcacucgaaaccguccagagguugcugccugugcugugccaagcgcacggacugacaccagagcaggucguggcaauugcgagcaacggagggggaaagcaggcacucgaaaccguccagagguugcugccugugcugugccaagcgcacggacuuacacccgaacaagucguggcaauugcgagccaugacgggggaaagcaggcacucgaaaccguccagagguugcugccugugcugugccaagcgcacggacuuacgccagagcaggucguggcaauugcgagcaacaucgggggaaagcaggcacucgaaaccguccagagguugcugccugugcugugccaagcgcacggacuaaccccagagcaggucguggcaauugugagcaacggagggggaaagcaggcacucgaaaccguccagagguugcugccugugcugugccaagcgcacggguugaccccagagcaggucguggcaauugcgagccaugacgggggaaagcaggcacucgaaaccguccagagguugcugccugugcugugccaagcgcacggccugaccccagagcaggucguggcaauugcgagccaugacgggggaaagcaggcacucgaaaccguccagagguugcugccugugcugugccaagcgcacggacugacaccagagcaggucguggcaauugcgagcaacggagggggaaagcaggcacucgaaaccguccagagguugcugccugugcugugccaagcgcacggccucaccccagagcaggucguggcaauugcgagccaugacgggggaaagcaggcacucgaaaccguccagagguugcugccugugcugugccaagcgcacggacuuacgccagagcaggucguggcaauugcgagcaacaucgggggaaagcaggcacucgaaaccguccagagguugcugccugugcugugccaagcgcacggacuaaccccagagcaggucguggcaauugcgagcaacggagggggaaagcaggcacucgaaaccguccagagguugcugccugugcugugccaagcgcacggguugaccccagagcaggucguggcaauugcgagccaugacgggggaaagcaggcacucgaaaccguccagagguugcugccugugcugugccaagcgcacggccugaccccagagcaggucguggcaauugcgagccaugacgggggaaagcaggcacucgaaaccguccagagguugcugccugugcugugccaagcgcacggacugacaccagagcaggucguggcaauugcgagcaacggagggggaaagcaggcacucgaaaccguccagagguugcugccugugcugugccaagcgcacggacucacgccugagcagguaguggcuauugcauccaacaacgggggcagacccgcacuggagucaaucguggcccagcuuucgaggccggaccccgcgcuggccgcacucacuaaugaucaucuuguagcgcuggccugccucggcggacgacccgccuuggaugcggugaagaaggggcucccgcacgcgccugcauugauuaagcggaccaacagaaggauucccgagaggacaucacaucgaguggcagguucccaacucgugaagagugaacuugaggagaaaaagucggagcugcggcacaaauugaaauacguaccgcaugaauacaucgaacuuaucgaaauugcuaggaacucgacucaagacagaauccuugagaugaagguaauggaguucuuuaugaagguuuauggauaccgagggaagcaucucgguggaucacgaaaacccgacggagcaaucuauacgguggggagcccgauugauuacggagugaucgucgacacgaaagccuacagcgguggguacaaucuucccaucgggcaggcagaugagaugcaacguuaugucgaagaaaaucagaccaggaacaaacacaucaauccaaaugagugguggaaaguguauccuucaucagugaccgaguuuaaguuuuuguuugucucugggcauuucaaaggcaacuauaaggcccagcucacacgguugaaucacauuacgaacugcaauggugcgguuuuguccguagaggaacugcucauugguggagaaaugaucaaagcgggaacucugacacuggaagaagucagacgcaaguuuaacaauggcgagaucaauuuccgcucauaaaaaaucagccucgacugugccuucuaguugccagccaucuguuguuugccccucccccgugccuuccuugacccuggaaggugccacucccacuguccuuuccuaauaaaaugaggaaauugcaucacaacacucaacccuaucucggucuauucuuuugauuuauaagggauuuugccgauuucggccuauugguuaaaaaaugagcugauuuaacaaaaauuuaacgcgaauuaauucuguggaaugugugucaguuaggguguggaaaguccccaggcuccccagcaggcagaaguaugcaaagcaugcaucucaauuagucagcaaccagguguggaaaguccccaggcuccccagcaggcagaaguaugcaaagcaugcaucucaauuagucagcaaccauagucccgccccuaacuccgcccaucccgccccuaacuccgcccaguuccgcccauucuccgccccauggcugacuaauuuuuuuuauuuaugcagaggccgaggccgccucugccucugagcuauuccagaaguagugaggaggcuuuuuuggaggc

SEQ ID NO: 18 - RNA, 3788 nt, Artificial Sequence
  CCR5-Uco-R mRNA
ggggccaccauggacuauaaggaccacgacggagacuacaaggaucaugauauugauuacaaagacgaugacgauaagauggccccaaagaagaagcggaaggucgguauccacggagucccagcagccguagauuugagaacuuugggauauucacagcagcagcaggaaaagaucaagcccaaagugaggucgacagucgcgcagcaucacgaagcgcuggugggucauggguuuacacaugcccacaucguagccuugucgcagcacccugcagcccuuggcacggucgccgucaaguaccaggacaugauugcggcguugccggaagccacacaugaggcgaucgucgguguggggaaacaguggagcggagcccgagcgcuugaggcccuguugacggucgcgggagagcugagagggccuccccuucagcuggacacgggccaguugcugaagaucgcgaagcggggaggagucacggcggucgaggcggugcacgcguggcgcaaugcgcucacgggagcaccccucaaccugaccccagagcaggucguggcaauugcgagcaacaucgggggaaagcaggcacucgaaaccguccagagguugcugccugugcugugccaagcgcacggacuuacgccagagcaggucguggcaauugcgagcaacaacgggggaaagcaggcacucgaaaccguccagagguugcugccugugcugugccaagcgcacggacuaaccccagagcaggucguggcaauugcgagcaacaucgggggaaagcaggcacucgaaaccguccagagguugcugccugugcugugccaagcgcacggguugaccccagagcaggucguggcaauugcgagcaacggagggggaaagcaggcacucgaaaccguccagagguugcugccugugcugugccaagcgcacggccugaccccagagcaggucguggcaauugcgagcaacaacgggggaaagcaggcacucgaaaccguccagagguugcugccugugcugugccaagcgcacggacugacaccagagcaggucguggcaauugcgagcaacggagggggaaagcaggcacucgaaaccguccagagguugcugccugugcugugccaagcgcacggacuuacacccgaacaagucguggcaauugcgagccaugacgggggaaagcaggcacucgaaaccguccagagguugcugccugugcugugccaagcgcacggacuuacgccagagcaggucguggcaauugcgagcaacaucgggggaaagcaggcacucgaaaccguccagagguugcugccugugcugugccaagcgcacggacuaaccccagagcaggucguggcaauugcgagcaaccacgggggaaagcaggcacucgaaaccguccagagguugcugccugugcugugccaagcgcacggguugaccccagagcaggucguggcaauugcgagcaacggagggggaaagcaggcacucgaaaccguccagagguugcugccugugcugugccaagcgcacggccugaccccagagcaggucguggcaauugcgagccaugacgggggaaagcaggcacucgaaaccguccagagguugcugccugugcugugccaagcgcacggacugacaccagagcaggucguggcaauugcgagcaacaucgggggaaagcaggcacucgaaaccguccagagguugcugccugugcugugccaagcgcacggccucaccccagagcaggucguggcaauugcgagcaacggagggggaaagcaggcacucgaaaccguccagagguugcugccugugcugugccaagcgcacggacuuacgccagagcaggucguggcaauugcgagcaaccacgggggaaagcaggcacucgaaaccguccagagguugcugccugugcugugccaagcgcacggacuaaccccagagcaggucguggcaauugcgagccaugacgggggaaagcaggcacucgaaaccguccagagguugcugccugugcugugccaagcgcacggguugaccccagagcaggucguggcaauugcgagcaacggagggggaaagcaggcacucgaaaccguccagagguugcugccugugcugugccaagcgcacggccugaccccagagcaggucguggcaauugcgagccaugacgggggaaagcaggcacucgaaaccguccagagguugcugccugugcugugccaagcgcacggacugacaccagagcaggucguggcaauugcgagcaacggagggggaaagcaggcacucgaaaccguccagagguugcugccugugcugugccaagcgcacggacucacgccugagcagguaguggcuauugcauccaacggagggggcagacccgcacuggagucaaucguggcccagcuuucgaggccggaccccgcgcuggccgcacucacuaaugaucaucuuguagcgcuggccugccucggcggacgacccgccuuggaugcggugaagaaggggcucccgcacgcgccugcauugauuaagcggaccaacagaaggauucccgagaggacaucacaucgaguggcagguucccaacucgugaagagugaacuugaggagaaaaagucggagcugcggcacaaauugaaauacguaccgcaugaauacaucgaacuuaucgaaauugcuaggaacucgacucaagacagaauccuugagaugaagguaauggaguucuuuaugaagguuuauggauaccgagggaagcaucucgguggaucacgaaaacccgacggagcaaucuauacgguggggagcccgauugauuacggagugaucgucgacacgaaagccuacagcgguggguacaaucuucccaucgggcaggcagaugagaugcaacguuaugucgaagaaaaucagaccaggaacaaacacaucaauccaaaugagugguggaaaguguauccuucaucagugaccgaguuuaaguuuuuguuugucucugggcauuucaaaggcaacuauaaggcccagcucacacgguugaaucacauuacgaacugcaauggugcgguuuuguccguagaggaacugcucauugguggagaaaugaucaaagcgggaacucugacacuggaagaagucagacgcaaguuuaacaauggcgagaucaauuuccgcucauaaaaaaucagccucgacugugccuucuaguugccagccaucuguuguuugccccucccccgugccuuccuugacccuggaaggugccacucccacuguccuuuccuaauaaaaugaggaaauugcaucacaacacucaacccuaucucggucuauucuuuugauuuauaagggauuuugccgauuucggccuauugguuaaaaaaugagcugauuuaacaaaaauuuaacgcgaauuaauucuguggaaugugugucaguuaggguguggaaaguccccaggcuccccagcaggcagaaguaugcaaagcaugcaucucaauuagucagcaaccagguguggaaaguccccaggcuccccagcaggcagaaguaugcaaagcaugcaucucaauuagucagcaaccauagucccgccccuaacuccgcccaucccgccccuaacuccgcccaguuccgcccauucuccgccccauggcugacuaauuuuuuuuauuuaugcagaggccgaggccgccucugccucugagcuauuccagaaguagugaggaggcuuuuuuggaggc

(19) United States
(12) Patent Application Publication
(10) Pub. No.: US 2017/0152496 A1
(43) Pub. Date: Jun. 1, 2017

(54) TAL-EFFECTOR NUCLEASE FOR TARGETED KNOCKOUT OF THE HIV CO-RECEPTOR CCR5

(71) Applicant: ADVANCED GENE & CELL TECHNOLOGIES LLC (AGCT LLC), Saint-Petersburg (RU)

(72) Inventors: Ulrike Mock, London (GB); Boris Fehse, Hamburg (DE)

(21) Appl. No.: 15/309,406

(22) PCT Filed: May 4, 2015
(86) PCT No.: PCT/DE2015/200295

§ 371 Date: Nov. 7, 2016

(30) Foreign Application Priority Data
May 7, 2014 (DE) 10 2014 106 327.9

(51) Int. Cl. C12N 9/22; C12N 15/11

(52) U.S. Cl. CPC C12N 9/22; C12N 15/111

(57) ABSTRACT

A novel TAL-effector nuclease (TALEN) for targeted knockout of the HIV co-receptor CCR5. One aspect provides a TAL-effector nuclease pair with a first and a second TAL-effector nuclease monomer, each TAL-effector nuclease monomer having an endonuclease domain having type II endonuclease activity and a TAL-effector DNA binding domain having a plurality of repeat units, each with a variable amino acid pair RVD, and wherein

a) the TAL-effector DNA binding domain of the first TAL-effector nuclease monomer binds to the target sequence GCTGGTCATCCTCATCCTG (SEQ ID NO: 1) and/or comprises the RVD sequence NH HD NG NH NH NG HD NI NG HD HD NG HD NI NG HD HD NG NN, and
b) the TAL-effector DNA binding domain of the second TAL-effector nuclease monomer binds to the target sequence AGATGTCAGTCATGCTCTT (SEQ ID NO: 2) and/or comprises the RVD sequence NI NN NI NG NN NG HD NI NH NG HD NI NG NH HD NG HD NG NG.

The invention relates to a novel TAL effector nuclease (TALEN) for targeted knockout of the HIV co-receptor CCR5.

In addition to its actual function in the cell, the chemokine receptor CCR5 plays an important role in HIV infection. Here, for what are known as the CCR5-tropic strains of the HI virus, it makes an appearance as a co-receptor which mediates the initial HIV infection. If no CCR5 is present on the surface of a T helper cell, the HI viruses cannot fuse with the host cell, and no infection occurs. Thus, a homozygous deletion (CCR5Δ32) in the CCR5 gene, which is present in approximately 1% of Western Europeans and “white” Americans (“Caucasians”), provides almost complete protection from an HIV infection with CCR-tropic strains. As a consequence, CCR5 is a very interesting target for HIV therapy.

Pharmacological approaches in the past which have been aimed at blockading CCR5 require life-long treatment in the context of combined antiretroviral therapy, ART. In the long term, this is associated with potentially severe side effects, and also with a lack of compliance by patients and with the development of resistance. On the other hand, a genetic deletion (“knockout”) of the CCR5 (in the context of gene therapy) would in the ideal case be sufficient as a single treatment because the genetic protection is passed on to all daughter cells. This is not only corroborated by the natural resistance of CCR5Δ32-homozygous individuals, but also by the case report of successful therapy of a HIV infection in what are known as the “Berlin patients” following allogenic stem cell transplantation with CCR5Δ32-homozygous donor cells (Hater G et al. Long-term control of HIV by CCRDelta32/Delta32 stem-cell transplantation. N Engl J Med. 2009, 360: 692-698; Allers K et al. Evidence for the cure of HIV infection by CCR5Δ32/Δ32 stem cell transplantation. Blood 2011; 117: 2791-2799).

Based on these observations, designs for genetic knockout of CCR5 in HIV patients were developed. The most promising strategies which are currently available are based on what are known as “designer nucleases” (see, for example, Manjunath N. et al., Newer Gene Editing Technologies toward HIV Gene Therapy, Viruses 2013, 5, 2748-2766). These designer nucleases consist of two components: a recognition domain, which determines the specificity in the genome and can be designed almost completely without constraints, and a nuclease domain, which induces a double-strand break at the selected site in the genome. By means of a defective repair of this double-strand break, the open reading frame of the target gene is displaced and thus, in the ideal case, a knockout is obtained. The first widely applicable designer nucleases were the zinc finger nucleases (ZFN). Sangamo BioSciences, Inc., for example, are currently testing a CCR5-specific zinc finger nuclease developed by them (http://www.sangamo.com/pipeline/sb-728.html) for clinical applications, using the description SB-728 (Tebas et al., Gene Editing of CCR5 in Autologous CD4 T Cells of Persons Infected with HIV. N Engl J Med 2014; 370:901-10). The clinical study demonstrated the feasibility of the approach, but long-term clinical effects on the virus load could only be observed in a volunteer, who proved to be heterozygous for the natural CCR5Δ32 mutation.

TAL effector nucleases (transcription activator-like effector nucleases, TALEN) are the next generation of designer nucleases (see, for example, Mussolino, C, Cathomen T. TALE nucleases: tailored genome engineering made easy, Curr Opin Biotechnol. 2012, 23(5): 644-50; WO 2011/072246 A2; EP 2510096 A2; WO 2011/154393 A1; WO 2011/159369 A1; WO 2012/093833 A2; WO 2013/182910 A2). Compared with ZFN, they exhibit a higher specificity, so that the risk of off-target effects, i.e. the appearance of mutations at a site in the genome other than the desired site, is substantially reduced (Händel E-M, Cathomen T. Zinc-finger nuclease based genome surgery: it's all about specificity. Curr Gene Ther 2011, 11: 28-37; Mussolino C et al. A novel TALE nuclease scaffold enables high genome editing activity in combination with low toxicity. Nucleic Acids Res. 2011; 39: 9283-9293).

CCR5-specific TALENs are already known (see, for example, Mussolino C et al. A novel TALE nuclease scaffold enables high genome editing activity in combination with low toxicity. Nucleic Acids Res. 2011; 39: 9283-9293; WO 2011/146121 A1; WO 2012/093833 A2; US 2013/0217131 A1), but until now, a clinical use has not been described.

Thus, there is still a need for means for the efficient treatment of a HIV infection. The object of the invention is therefore to provide such a means. In particular, the object of the present invention is to provide a means with the aid of which a more efficient knockout of the HIV co-receptor CCR5 can be obtained than previously.

The object is achieved by means of the subject matter of claim 1 and the other subordinate claims. Appropriate embodiments of the solution in accordance with the invention are provided in the dependent claims.

In a first aspect, the invention provides a TAL effector nuclease pair, comprising a first and a second TAL effector nuclease monomer, wherein each TAL effector nuclease monomer comprises an endonuclease domain with Type II endonuclease activity and a TAL effector DNA-binding domain with a plurality of repeats, each having a variable amino acid pair RVD, and wherein

a) the TAL effector DNA-binding domain of the first TAL effector nuclease monomer binds to the target sequence GCTGGTCATCCTCATCCTG (SEQ ID NO: 1) and/or comprises the RVD sequence NH HD NG NH NH NG HD NI NG HD HD NG HD NI NG HD HD NG NN, and
b) the TAL effector DNA-binding domain of the second TAL effector nuclease monomer binds to the target sequence AGATGTCAGTCATGCTCTT (SEQ ID NO: 2) and/or comprises the RVD sequence NI NN NI NG NN NG HD NI NH NG HD NI NG NH HD NG HD NG NG.

The TAL effector nuclease pair in accordance with the invention is capable of causing a knockout of the CCR5 in primary T lymphocytes with an as yet unattained high efficiency of >50%. In this regard, the invention also surprisingly allows a consistent biallelic knockout of both CCR5 alleles, and thus complete protection of the modified cells before HIV entry, in contrast to the opinion expressed by leading experts in the prior art that this is not currently possible (“Consistent nuclease-mediated biallelic knockdown is not yet tenable”, see Kay, M. A. and Walker, B. D., 2014, Engineering Cellular Resistance to HIV, N Engl J Med 370:968-969). Moreover, it has been shown that the TALEN pair in accordance with the invention is extraordinarily suitable for a gene transfer based on mRNA transfection (and thus particularly gentle, safe and GMP-compatible). In this manner, the invention in the first place provides a means based on a designer nuclease for HIV treatment which has a high knockout efficiency and selectivity with low off-target effects and other pharmacologically advantageous properties.

The term “TAL effector nuclease” or “TALEN” (transcription activator-like effector nuclease) should be understood here to mean a fusion protein which contains a DNA-binding domain of a TAL effector (TALE) and a DNA cleavage domain of a restriction endonuclease. TAL effectors are DNA-binding proteins which are formed from plant pathogens such as Xanthomonas spp. DNA binding of the TAL effectors is mediated via a domain with a variable number (as a rule 5 to 30) of repeat units (“repeats”), usually formed from 33 to 35 amino acids. Each of these repeats has two highly variable amino acid residues (repeat variable diresidue, RVD), as a rule at positions 12 and 13, which bind to exactly one base of a DNA target sequence. The relationship between RVD and DNA target nucleotide is given below.

| RVD | RVD | |
|---|---|---|
| (single | (three- | Nucleotide(s) |
| letter code) | letter code) | |
| NH | Asn-His | G |
| HD | His-Asp | C |
| NG | Asn-Gly | T |
| NI | Asn-Ile | A |
| NN | Asn-Asn | R (G, A) |
| NK | Asn-Lys | G |
| NS | Asn-Ser | N (A, C, G, T) |

The term “RVD sequence” as used here should be understood to mean a contiguous sequence of RVDs in a TAL effector binding domain, wherein the sequence here, unless otherwise stated, is given in the N-C direction, i.e. from the N end to the C end. Clearly, the person skilled in the art will be aware here that the RVDs of a “RVD sequence” do not follow each other directly insofar as they are not themselves directly covalently connected together, but the repeats in which the RVDs are contained are connected together directly so that the respective RVDs are separated by amino acids of the basic structure of the repeats.

The term “target sequence” should be understood here to mean a nucleotide sequence, as a rule a DNA sequence, to which the TAL effector binding domain binds.

The RVD sequences disclosed here, consisting of 19 RVDs, have the following target sequences (in the 5′-3′ direction; single letter codes for the amino acids):

(SEQ ID NO: 1)
NH HD NG NH NH NG HD NI NG HD HD NG HD NI NG HD
HD NG NN GCTGGTCATCCTCATCCTG
(SEQ ID NO: 2)
NI NN NI NG NN NG HD NI NH NG HD NI NG NH HD NG
HD NG NG AGATGTCAGTCATGCTCTT

The term “repeat” in respect of a TAL effector binding domain as used here should be understood to mean a contiguous sequence, as a rule of 33-35, usually 34 amino acids which, apart from the highly variable RVDs at positions 12 and 13, have a substantially identical amino acid sequence. It is also possible that within the conserved basic structure of the repeat, i.e. the essentially preserved structure into which the highly variable RVDs are embedded, individual amino acids might vary, for example at positions 4, 10 and/or 32 in a repeat formed from 34 amino acids. A typical repeat may, for example, have the following amino acid sequence (suffixes provide the position within the repeat):

(SEQ ID NO: 5)
LTPX₄QVVAIX₁₀SX₁₂X₁₃GGKQALETVQRLLPVLCQX₃₂HG

X represents any amino acid, wherein at positions 12 and 13 the hypervariable amino acids of the RVDs are placed. At position 4 (X4), for example, the amino acids E, Q, D or A may be positioned; the amino acids A or D are at position 32 (X32). A or V may be positioned at position 10, for example. Examples of repeats are given below (XX represents the hypervariable amino acids of the RVDs; variable amino acids are underlined to highlight them):

(SEQ ID NO: 6)
LTPEQVVAIASXXGGKQALETVQRLLPVLCQAHG
(SEQ ID NO: 7)
LTPQQVVAIASXXGGKQALETVQRLLPVLCQAHG
(SEQ ID NO: 8)
LTPDQVVAIASXXGGKQALETVQRLLPVLCQDHG
(SEQ ID NO: 9)
LTPAQVVAIASXXGGKQALETVQRLLPVLCQDHG
(SEQ ID NO: 10)
LTPEQVVAIVSXXGGKQALETVQRLLPVLCQAHG

The TAL effector binding domain may contain one or more of such variations of repeats, but may also include a mixture of different variations.

The outer repeat immediately adjacent to the nuclease domain may comprise fewer amino acids, for example only the first 15, 16, 17, 18, 19 or 20 amino acids of the other repeats. A repeat of this type is also known as a “half repeat”.

The term “DNA-binding domain” as used here should be understood to mean a region of a protein which induces binding of the protein to a DNA. In the case of a DNA-binding domain of a TAL effector, this occurs by means of the repeats described in more detail above.

The wording wherein a TAL effector DNA-binding domain is said to “bind” to a DNA sequence should be understood to mean that the TAL effector DNA-binding domain binds specifically to the target sequence because of its RVD sequence. In this respect, it is not necessary, although it is preferred, for each nucleotide of the target sequence to be associated with a RVD in the binding domain. The relationship between the RVD sequence of the TAL effector DNA-binding domain and the target sequence must solely be such that specific binding to the target sequence occurs. “Specifically” in this context means that binding occurs essentially exclusively at the target sequence.

The term “TAL effector nuclease monomer” should be understood to mean a TAL effector nuclease which consists of a single polypeptide chain. The term “TAL effector nuclease pair” or “TALEN pair” should be understood to be a TALEN composed of two TAL effector nuclease monomers. The monomers represent a left or right arm of a TALEN, which bind to the opposing strands of a DNA and together carry out cleavage of the DNA at one site.

When a “left” or “right” TALEN or a “left” or “right” TALEN “arm” is mentioned in relation to a TALEN pair, this reflects the fact that in a TALEN pair, TALEN monomers are used in pairs, i.e. induce a strand break within a double-stranded DNA, wherein one monomer binds a target sequence on the sense strand, while another TALEN monomer of the TALEN pair binds a target sequence on the complementary antisense strand, and in fact so that the nuclease domains are oriented with respect to each other in a common region of DNA known as a “spacer” between the target sequences and here each cause a single strand break. “Left” and “right” TALEN monomers are thus the parts of a TALEN pair of this type, wherein the designation “left” TALEN is often assigned to the TALEN which binds to the sense strand, while the “right” TALEN binds the complementary strand. When a “left” or “right” TALEN is mentioned here, however, this does not specifically assign the “left” TALEN to the sense strand and the “right” TALEN to the complementary strand thereto.

Thus, the present invention also concerns a “TALEN pair”, i.e. a pair formed from two monomers in accordance with the invention, which respectively represent a left or right arm of a TALEN. In this regard, the present invention also concerns a TAL effector nuclease pair comprising a TAL effector nuclease monomer the TAL effector DNA-binding domain of which binds to the target sequence GCTGGTCATCCTCATCCTG (SEQ ID NO: 1) and/or comprises the RVD sequence NH HD NG NH NH NG HD NI NG HD HD NG HD NI NG HD HD NG NN, and a TAL effector nuclease monomer the TAL effector DNA-binding domain of which binds to the target sequence AGATGTCAGTCATGCTCTT (SEQ ID NO: 2) and/or comprises the RVD sequence NI NN NI NG NN NG HD NI NH NG HD NI NG NH HD NG HD NG NG.

The term “endonuclease domain with type II endonuclease activity” should be understood to mean a polypeptide which exhibits the DNA cleavage activity of a restriction endonuclease and cleaves the DNA within or in the immediate vicinity of the recognition sequence, requires no ATP and has no methyltransferase activity. The term “endonuclease domain with type IIS endonuclease activity” should be understood to mean a domain of a type II endonuclease with a cleavage site in the immediate vicinity of the recognition sequence, but not within it.

The term “CCR5” as used here should be understood to mean CC chemokine receptor type 5 (also denoted CD195, CMKBR5 or CC-CKR5). A sequence for human CCR5 is provided in SEQ ID NO: 11 (see NCBI accession number NC_018914.2).

The term “vector” as used here should be understood to mean a transport vehicle for transferring a (usually foreign) nucleic acid into a living receptor cell by transfection or transduction. The term “gene transfer vector” as used here should be understood to mean a vector with the aid of which a gene can be introduced into a cell. (Gene transfer) vectors are well known to the person skilled in the art. Examples of gene transfer vectors are plasmids, viral vectors or mRNA.

The term “nucleic acid” should be understood to mean a polymer with nucleotides as the monomers. A nucleotide is a compound formed from a sugar residue, a nitrogen-containing heterocyclic organic base (nucleotide or nucleobase) and a phosphate group. The sugar residue is usually a pentose, deoxyribose in the case of DNA, ribose in the case of RNA. The nucleotides are linked together via the phosphate group by means of a phosphodiester bridge, as a rule between the 3′ C atom of the sugar component of a nucleoside (compound of nucleobase and sugar) and the 5′ C atom of the sugar component of the next nucleoside. The term “nucleic acid” includes, for example, DNA, RNA and mixed DNA/RNA sequences. The term “nucleic acid” as used here in particular means an isolated nucleic acid. The term “isolated nucleic acid” should be understood to mean a natural nucleic acid which has been liberated from its natural or original environment, or a synthetically produced nucleic acid.

The term “comprises” as used here defines both an item which exclusively exhibits the features grouped under the term, and also an item which has these features grouped under the term, along with more features. The definition of an item which states that it comprises specific features thus also includes the definition of that item by the definitive listing of these features, i.e. by the presence of these features alone.

In a preferred embodiment of the TAL effector nuclease pair in accordance with the invention, the endonuclease domain in each of the TAL effector nuclease monomers is C-terminal with respect to the TAL effector DNA-binding domain. Preferably, each repeat with the exception of the repeat immediately adjacent to the endonuclease domain comprises 33 to 35 amino acids, preferably 34 amino acids, wherein the RVDs are in positions 12 and 13 in each repeat. Particularly preferably, all of the repeats apart from the “half repeat” have the amino acid sequence of SEQ ID NO: 5, wherein E, Q, D or A may be in position 4; A or V may be in position 10, and A or D may be in position 32. The basic structure for the repeats may be identical or different for all of the repeats. The amino acids in one or more repeats may vary at positions within the basic structure, for example in positions 4, 10 and/or 32. The repeat which is immediately adjacent to the endonuclease domain may comprise a smaller number of amino acids, for example 15, 16, 17, 18, 19 or 20 amino acids, wherein in this case, the amino acids correspond to the first 15, 16, 17, 18, 19 or 20 amino acids of the other repeats. As an example in this regard, the amino acid at position 4 may be different; for example, it may be E, Q, D or A, and/or the amino acid at position 10 may be different, for example V instead of A.

Particularly preferably, the endonuclease domain of the TAL effector nuclease monomer is a type IIS endonuclease domain, particularly preferably the DNA cleavage domain of FokI endonuclease. An amino acid sequence for a suitable FokI cleavage domain is provided in SEQ ID NO: 12. However, other type II endonuclease cleavage domains may be considered. Type II endonucleases are known to the person skilled in the art, and suitable cleavage domains may be determined by means of routine investigations.

In a particularly preferred embodiment, the first TAL effector nuclease monomer comprises an amino acid sequence in accordance with SEQ ID NO: 3 and the second TAL effector nuclease monomer comprises an amino acid sequence in accordance with SEQ ID NO: 4. In this regard, SEQ ID NO: 3 provides the left TALEN (hereinafter also denoted CCR5-Uco-L or left arm of CCR5-Uco) and in SEQ ID NO: 4 the right TALEN (hereinafter also denoted CCR5-Uco-R or right arm of CCR5-Uco) of a TALEN pair which together cause a double-strand break in the DNA sequence of the CCR5 within the spacers between the target sequences in accordance with SEQ ID NO: 1 and SEQ ID NO: 2. Repair of this double-strand break by cellular repair systems (non-homologous end-joining, NHEJ) results in a high probability of a displacement of the reading frame and thus a knockout of CCR5.

In a second aspect, the present invention also relates to a nucleic acid comprising:

a) a first nucleic acid which codes for a first TAL effector nuclease monomer, wherein the first TAL effector nuclease monomer comprises an endonuclease domain with Type II endonuclease activity and a TAL effector DNA-binding domain with a plurality of repeats, each having a variable amino acid pair RVD, and wherein the TAL effector DNA-binding domain binds to the target sequence GCTGGTCATCCTCATCCTG (SEQ ID NO: 1) and/or comprises the RVD sequence NH HD NG NH NH NG HD NI NG HD HD NG HD NI NG HD HD NG NN, and
b) a second nucleic acid which codes for a second TAL effector nuclease monomer, wherein the second TAL effector nuclease monomer comprises an endonuclease domain with Type II endonuclease activity and a TAL effector DNA-binding domain with a plurality of repeats, each having a variable amino acid pair RVD, and wherein the TAL effector DNA-binding domain binds to the target sequence AGATGTCAGTCATGCTCTT (SEQ ID NO: 2) and/or comprises the RVD sequence NI NN NI NG NN NG HD NI NH NG HD NI NG NH HD NG HD NG NG.

In this aspect of the invention, the TALEN monomers forming the TALEN pair in accordance with the invention are coded together in a common nucleic acid. An example of the nucleic acid may be a plasmid or another suitable (gene transfer) vector. Suitable vectors as well as methods for their manufacture and their use are well known in the prior art. If appropriate, in addition to the TALEN code, the nucleic acid may also contain other elements, for example one or more promoters, as well as polyadenylation signals, etc.

The TALEN monomers forming the TALEN pair in accordance with the invention may also be coded separately in two nucleic acids. In a third aspect, the present invention thus also concerns a nucleic acid composition, comprising

a) a first nucleic acid which codes for a first TAL effector nuclease monomer, wherein the first TAL effector nuclease monomer comprises an endonuclease domain with Type II endonuclease activity and a TAL effector DNA-binding domain with a plurality of repeats, each having a variable amino acid pair RVD, and wherein the TAL effector DNA-binding domain binds to the target sequence GCTGGTCATCCTCATCCTG (SEQ ID NO: 1) and/or comprises the RVD sequence NH HD NG NH NH NG HD NI NG HD HD NG HD NI NG HD HD NG NN, and
b) a second nucleic acid which codes for a second TAL effector nuclease monomer, wherein the second TAL effector nuclease monomer comprises an endonuclease domain with Type II endonuclease activity and a TAL effector DNA-binding domain with a plurality of repeats, each having a variable amino acid pair RVD, and wherein the TAL effector DNA-binding domain binds to the target sequence AGATGTCAGTCATGCTCTT (SEQ ID NO: 2) and/or comprises the RVD sequence NI NN NI NG NN NG HD NI NH NG HD NI NG NH HD NG HD NG NG.

Preferably, the first and second nucleic acid are respectively a mRNA, particularly preferably a stabilized mRNA (see, for example, Kallen K.-J. et al., A novel, disruptive vaccination technology, Hum Vaccin Immunother. Oct. 1, 2013; 9(10): 2263-2276, doi: 10.4161/hv.25181; Kallen K.-J. and The β A., A development that may evolve into a revolution in medicine: mRNA as the basis for novel, nucleotide-based vaccines and drugs, Ther Adv Vaccines. January 2014; 2(1): 10-31, doi: 10.1177/2051013613508729). If appropriate, the first and second nucleic acid may also contain further elements, for example one or more promoters, as well as polyadenylation signals, etc., in addition to the TALEN code. Examples of suitable mRNAs for the left and right arm of a TALEN in accordance with the invention are given in SEQ ID NO: 17 and 18.

In the case of a mRNA, it is transported into the target cell(s), for example T lymphocytes, particularly preferably by means of the method described by Berdien et al. (Berdien B et al., TALEN-mediated editing of endogenous T-cell receptors facilitates efficient reprogramming of T lymphocytes by lentiviral gene transfer, Gene Therapy, 2014, doi:10.1038/gt.2014.26). Particularly preferably, both TALEN arms (right and left arm) are brought into a cell simultaneously.

Introducing the TALEN pair in accordance with the invention via mRNA enjoys a series of decisive advantages for clinical applications. The use of a DNA-based gene transfer vector can be avoided, which considerably simplifies the production and practical application. The mRNA-mediated expression of the TALEN occurs comparatively temporary, since the mRNA is rapidly degraded in the target cell. In this manner, the risk of off-target effects is further reduced. Moreover, the target cells only have to be cultured for a very brief period in vitro. GMP requirements can readily be complied with by the corresponding technology. In contrast to viral or plasmid vectors, no side effects due to the gene transfer itself (insertion mutagenesis for example) or due to a long-duration TALEN expression (off-target effects, activation of a TALEN-specific immune response) are anticipated as a result of undesirable vector insertion.

In further aspects, the present invention also relates to a vector, in particular a gene transfer vector, comprising a nucleic acid in accordance with the invention, and an isolated host cell, comprising a vector in accordance with the invention, a nucleic acid in accordance with the invention or a nucleic acid composition, wherein the isolated host cell is not a germ line cell of a human being, in particular not a human gamete or human embryonic gamete, and wherein it is not a human embryonic stem cell which has been obtained or is obtained by destroying a human embryo.

In a further aspect, the present invention relates to a pharmaceutical composition comprising a nucleic acid, nucleic acid composition or a vector in accordance with the present invention. The pharmaceutical composition may comprise adjuvants, for example solvents, solubilizers, solution accelerators, salt-forming agents, salts, buffers, viscosity and consistency adjusting agents, gelling agents, emulsifiers, solubilizers, wetting agents, spreading agents, antioxidants, preservatives, fillers and substrates, etc.

In a yet still further aspect, the present invention relates to a medicament comprising a nucleic acid, nucleic acid composition, a vector or a pharmaceutical composition in accordance with the present invention.

The invention will now be described in more detail with the aid of exemplary embodiments and the accompanying drawings, provided for illustrative purposes.

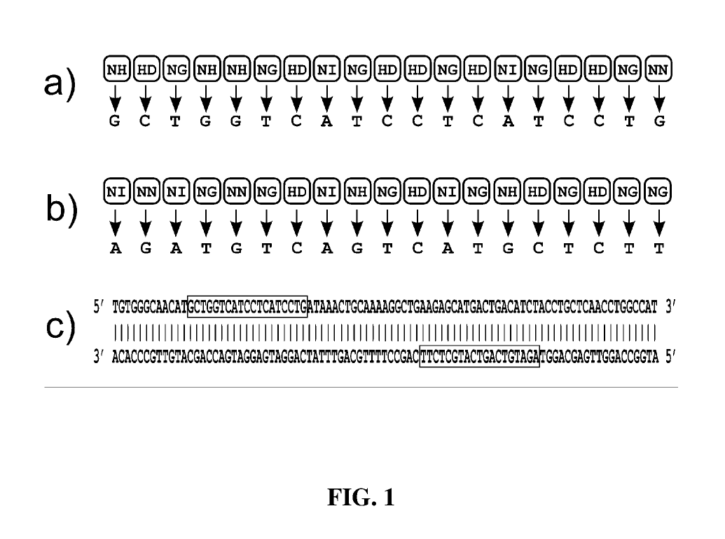

FIG. 1: A diagrammatic illustration of the DNA-binding domains of a CCR5-specific TALEN pair (“CCR5-Uco”) and its target sequences in the CCR5 gene. The respective lower lines show the target sequences for a) the left and b) the right TALEN arm; the respective top lines show the relevant RVDs (repeat variable di-residues) of the corresponding tale monomers in the boxes (amino acids are given in the single letter code); c) section (nt 135-221 of the sequence of SEQ ID NO: 11) of the CCR5 DNA with complementary strand. The target sequences for the left (top) and right (bottom, on complementary strand) arms of the CCR5-Uco-TALEN are highlighted by being framed with a box.

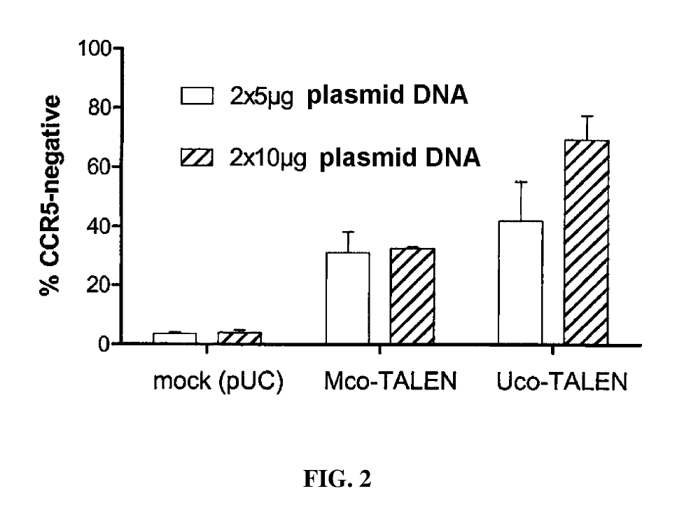

FIG. 2: Efficiency comparison between the CCR5 TALEN (“Uco”) in accordance with the invention and a control CCR5 TALEN (“Mco) from the prior art. Testing was carried out by plasmid transfection into a CCR5-positive, 293T cell-based reporter cell line. For all tested constructs, comparable transfection efficiencies were observed (by means of co-transfection by eGFP). CCR5 knockout was assayed 6 days after transfection of the CCR5+293T cell clone with the aid of a specific (anti-CD195-APC-Cy-7 antibody) (n=2). For the “mock” control, the cells were transfected with an irrelevant control plasmid (pUC), which did not code for any TALEN.

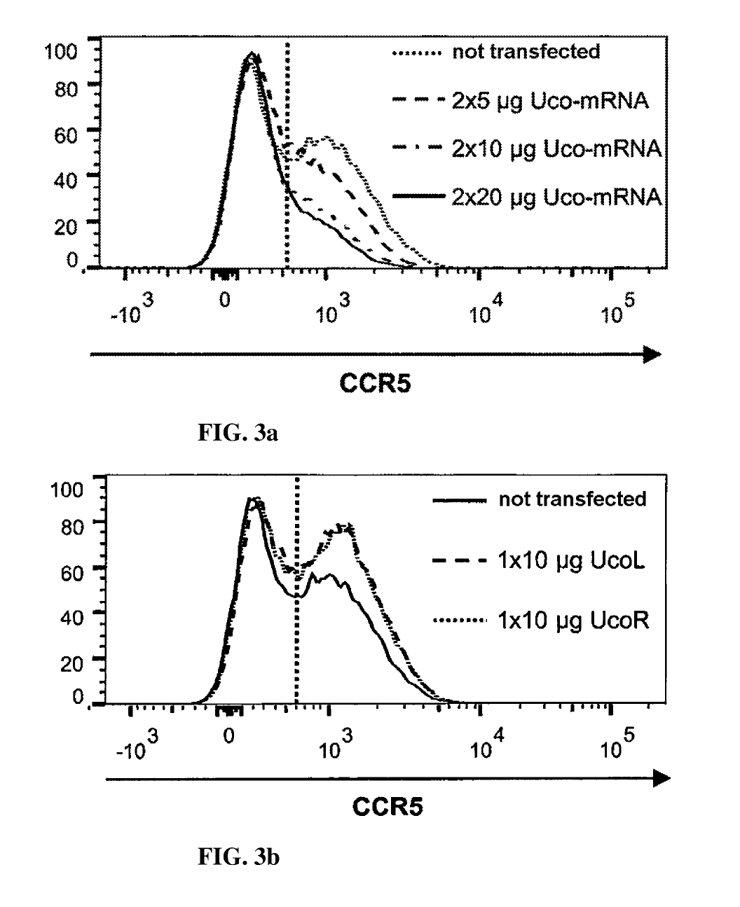

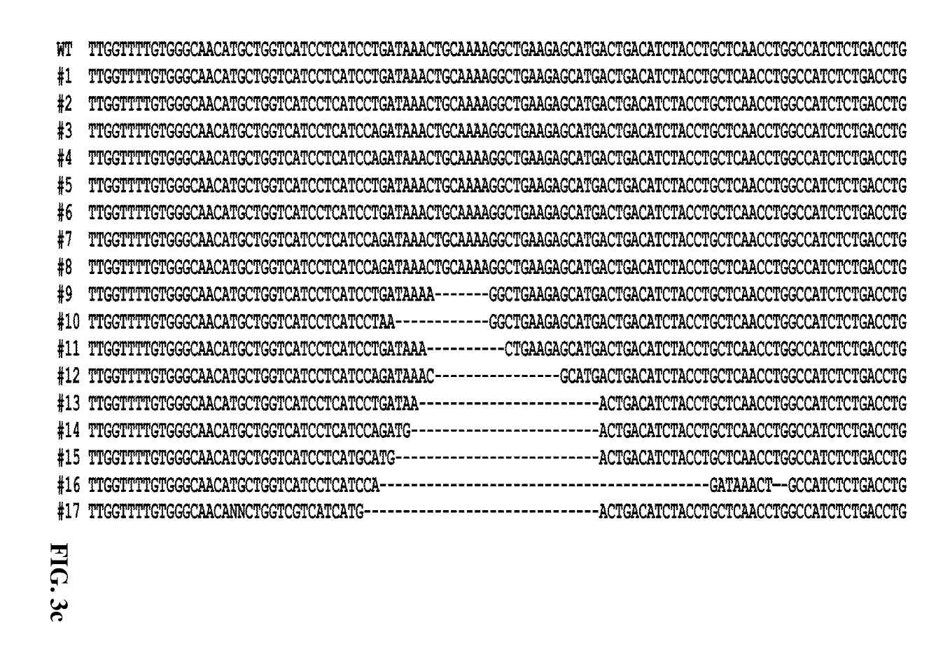

FIG. 3: CCR5 knockout in primary T lymphocytes with CCR5 Uco following mRNA transfection. After ex-vivo activation, approximately half of the primary T lymphocytes of a healthy volunteer expressed CCR5 (=cells to the right of the dashed line, see “not transfected”). a) After transfection of the CCR5-specific TALEN (Uco), the proportion of CCR5-positive cells reduced with increasing quantities of transfected mRNA (inverse proportion). b) The control transfection of the individual TALEN arms, on the other hand, did not result in a reduction in the proportion of CCR5-positive cells. CCR5 knockout was determined 6 days after the mRNA transfection with the aid of a specific antibody (anti-CD195-PerCP-Cy5.5 antibody). c) An analysis of the target site of the CCR5-Uco-TALEN exhibited a genetic knockout in 9 out of 17 (>50%) of the analysed primary T lymphocytes.

### EXAMPLES

A CCR5-specific TALEN (hereinafter “CCR5-Uco”) in accordance with the invention was produced and investigated. The TALEN in accordance with the invention differs from TALENs which have been described before as regards the target sequence in the CCR5 gene (see FIG. 1) recognized by it.

In contrast to a codon-optimized CCR5 TALEN (“Mco”; see SEQ ID NO: 13, 14), based on published work from the laboratory of Prof. Toni Cathomen (Freiburg) (see Mussolino C et al., A novel TALE nuclease scaffold enables high genome editing activity in combination with low toxicity, Nucleic Acids Res. 2011, 39: 9283-9293), the CCR5-Uco-TALEN in accordance with the invention exhibited a significantly higher rate of induction of CCR5 knockout after plasmid transfection into a reporter cell line (see FIG. 2). The nucleic acid sequence of the TALEN components codon-optimized for use in human cells was in this case based on the publications from Feng Zhang's group (Zhang et al., Efficient construction of sequence-specific TAL effectors for modulating mammalian transcription, Nature Biotechnology, 2011, 29, 149-153; Sanjana N E et al., A TAL Effector Toolbox for Genome Engineering, Nature Protocols, 2012, 7: 171-192).

The RVD sequences of the CCR5-Mco-TALEN in accordance with the prior art are as follows:

Left arm (L) = NN NG NN NN NN HD NI NI HD NI NG
NN HD NG NN NN NG HD;
Right arm (R) = HD NG NG HD NI NN HD HD NG NG NG
NG NN HD NI NN NG NG.

The associated DNA recognition sequence:

L on sense strand =
(SEQ ID NO: 15)
GTGGGCAACATGCTGGTC;
R on antisense strand =
(SEQ ID NO: 16)
CTTCAGCCTTTTGCAGTT.

The length of the spacer was 15 nt. Here again, the production of the TALEN plasmids was based on the publications by Zhang F, or Sanjana N E et al. (see above).

Using mRNA transfection (see Berdien B et al., TALEN-mediated editing of endogenous T-cell receptors facilitates efficient reprogramming of T lymphocytes by lentiviral gene transfer, Gene Therapy, 2014, doi:10.1038/gt.2014.26) with the CCR5-Uco in accordance with the invention, a CCR5 knockout was brought about in primary T lymphocytes (see FIG. 3). Nucleic acid sequences for the mRNA used in this regard are provided in SEQ ID NO: 17 and 18. SEQ ID NO: 17 shows the mRNA for the left TALEN arm; SEQ ID NO: 18 shows the mRNA for the right TALEN arm. The nucleotides 10-3225 of the mRNAs in SEQ ID NO: 17 and 18 respectively code for the TALEN arms (monomers); their amino acid sequences are given in SEQ ID NO: 3 and 4.

The transfected mRNA was produced via the T7 promoter following AvrII linearization of the vector. Since the AvrII cleavage site lies 563 bp behind the stop codon, the given sequence is longer than the open reading frame. After linearization, the respective Uco TALEN DNA was used as the template for the production of the mRNA using the T7 mScript™ Standard mRNA Production System from Cellscript (Madison, Wis. 53713 USA). According to the manufacturer's instructions, the mRNA was provided with a 5′ cap and a poly-A tail. Transfection of the mRNA was carried out by electroporation of the primary T cells for 10 ms at 300 V. In contrast, it was not possible to obtain a CCR5 knockout in primary T cells or Z cell lines with the Mco-CCR5 TALEN (although a k.o. of the T cell receptor was possible, see Berdien et al, 2014, see above). It was only possible to knock out a significant proportion of >50% of the CCR5 alleles by means of mRNA transfer using the CCR5-Uco TALEN in accordance with the invention (FIG. 3c). It is clear from this that only sufficiently active TALENs are able to carry out their function in primary T cells following a mRNA transfection. Thus, the CCR5 TALEN in accordance with the invention is particularly suitable for use via mRNA transfection, making it extremely attractive for clinical application.

Overview of Sequences:

| SEQ ID NO: | Type | Description |
|---|---|---|
| 01 | DNA | Target sequence TALEN CCR5-Uco L |
| 02 | DNA | Target sequence TALEN CCR5-Uco R |
| 03 | PRT | TALEN CCR5-Uco L |
| 04 | PRT | TALEN CCR5-Uco R |
| 05 | PRT | Repeat sequence (consensus) |
| 06 | PRT | Repeat sequence |
| 07 | PRT | Repeat sequence |
| 08 | PRT | Repeat sequence |
| 09 | PRT | Repeat sequence |
| 10 | PRT | Repeat sequence |
| 11 | DNA | hCCR5 |
| 12 | PRT | FokI cleavage domain |
| 13 | PRT | TALEN CCR5-Mco L |
| 14 | PRT | TALEN CCR5-Mco R |
| 15 | DNA | Target sequence TALEN CCR5-Mco L |
| 16 | DNA | Target sequence TALEN CCR5-Mco R |
| 17 | mRNA | mRNA CCR5-Uco L |
| 18 | mRNA | mRNA CCR5-Uco R |

### SEQUENCE LISTING—FREE TEXT

TALEN repeat
Any amino acid, or E, Q, D or A
Any amino acid, or A or V
Any amino acid, or A or D
Repeat variable diresidue (RVD)
FokI cleavage domain

1. A TAL-effector nuclease pair, comprising a first and a second TAL-effector nuclease monomer, wherein each TAL-effector nuclease monomer comprises an endonuclease domain with Type II endonuclease activity and a TAL-effector DNA-binding domain with a plurality of repeats, each having a variable amino acid pair RVD, and wherein
a) the TAL-effector DNA-binding domain of the first TAL-effector nuclease monomer binds to the target sequence GCTGGTCATCCTCATCCTG (SEQ ID NO: 1) and/or comprises the RVD sequence NH HD NG NH NH NG HD NI NG HD HD NG HD NI NG HD HD NG NN,
and
b) the TAL-effector DNA-binding domain of the second TAL-effector nuclease monomer binds to the target sequence AGATGTCAGTCATGCTCTT (SEQ ID NO: 2) and/or comprises the RVD sequence NI NN NI NG NN NG HD NI NH NG HD NI NG NH HD NG HD NG NG.

2. The TAL-effector nuclease pair as claimed in claim 1, wherein the endonuclease domain in each of the TAL-effector nuclease monomers is C-terminal with respect to the TAL-effector DNA-binding domain and each repeat with the exception of that immediately adjacent to the endonuclease domain comprises 33 to 35 amino acids, wherein the RVDs are in positions 12 and 13 in each repeat.

3. The TAL-effector nuclease pair as claimed in claim 1, wherein the endonuclease domain of the TAL-effector nuclease monomer is a DNA cleavage domain of FokI endonuclease.

4. The TAL-effector nuclease pair as claimed in claim 1, wherein the first TAL-effector nuclease monomer comprises an amino acid sequence in accordance with SEQ ID NO: 3 and the second TAL-effector nuclease monomer comprises an amino acid sequence in accordance with SEQ ID NO: 4.

5. A nucleic acid comprising:
a) a first nucleic acid which codes for a first TAL-effector nuclease monomer, wherein the first TAL-effector nuclease monomer comprises an endonuclease domain with Type II endonuclease activity and a TAL-effector DNA-binding domain with a plurality of repeats, each having a variable amino acid pair RVD, and wherein the TAL-effector DNA-binding domain binds to the target sequence GCTGGTCATCCTCATCCTG (SEQ ID NO: 1) and/or comprises the RVD sequence NH HD NG NH NH NG HD NI NG HD HD NG HD NI NG HD HD NG NN, and
b) a second nucleic acid which codes for a second TAL-effector nuclease monomer, wherein the second TAL-effector nuclease monomer comprises an endonuclease domain with Type II endonuclease activity and a TAL-effector DNA-binding domain with a plurality of repeats, each having a variable amino acid pair RVD, and wherein the TAL-effector DNA-binding domain binds to the target sequence AGATGTCAGTCATGCTCTT (SEQ ID NO: 2) and/or comprises the RVD sequence NI NN NI NG NN NG HD NI NH NG HD NI NG NH HD NG HD NG NG.

6. A vector comprising a nucleic acid as claimed in claim 5.

7. A nucleic acid composition comprising:
a) first nucleic acid which codes for a first TAL-effector nuclease monomer, wherein the first TAL-effector nuclease monomer comprises an endonuclease domain with Type II endonuclease activity and a TAL-effector DNA-binding domain with a plurality of repeats, each having a variable amino acid pair RVD, and wherein the TAL-effector DNA-binding domain binds to the target sequence GCTGGTCATCCTCATCCTG (SEQ ID NO: 1) and/or comprises the RVD sequence NH HD NG NH NH NG HD NI NG HD HD NG HD NI NG HD HD NG NN, and
b) a second nucleic acid which codes for a second TAL-effector nuclease monomer, wherein the second TAL-effector nuclease monomer comprises an endonuclease domain with Type II endonuclease activity and a TAL-effector DNA-binding domain with a plurality of repeats, each having a variable amino acid pair RVD, and wherein the TAL-effector DNA-binding domain binds to the target sequence AGATGTCAGTCATGCTCTT (SEQ ID NO: 2) and/or comprises the RVD sequence NI NN NI NG NN NG HD NI NH NG HD NI NG NH HD NG HD NG NG.

8. The nucleic acid composition as claimed in claim 7, wherein the first nucleic acid is a first mRNA and the second nucleic acid is a second mRNA.

9. The nucleic acid composition as claimed in claim 8, wherein the first nucleic acid comprises a sequence in accordance with SEQ ID NO: 17 and the second nucleic acid comprises a sequence in accordance with SEQ ID NO: 18.

10. An isolated host cell comprising a nucleic acid as claimed in claim 5, with the proviso that the host cell is not a human gamete or human embryonic gamete, and is not a human embryonic stem cell which has been obtained or is obtained by destroying a human embryo.

11. A pharmaceutical composition comprising a nucleic acid as claimed in claim 5.

12. A medicament comprising a nucleic acid as claimed in claim 5.

13. The TAL-effector nuclease pair as claimed in claim 1, wherein the endonuclease domain in each of the TAL-effector nuclease monomers is C-terminal with respect to the TAL-effector DNA-binding domain and each repeat with the exception of that immediately adjacent to the endonuclease domain comprises 34 amino acids, wherein the RVDs are in positions 12 and 13 in each repeat.

14. An isolated host cell comprising a nucleic acid composition as claimed in claim 7, with the proviso that the host cell is not a human gamete or human embryonic gamete, and is not a human embryonic stem cell which has been obtained or is obtained by destroying a human embryo.

15. An isolated host cell comprising a vector as claimed in claim 6, with the proviso that the host cell is not a human gamete or human embryonic gamete, and is not a human embryonic stem cell which has been obtained or is obtained by destroying a human embryo.

16. A pharmaceutical composition comprising a nucleic acid composition as claimed in claim 7.

17. A pharmaceutical composition comprising a vector as claimed in claim 6.

18. A medicament comprising a nucleic acid as claimed in claim 7.

19. A medicament comprising a vector as claimed in claim 6.

20. A medicament comprising a pharmaceutical composition as claimed in claim 11.
